(19) United States
(12) Patent Application Publication
(10) Pub. No.: US 2006/0116640 A1
(43) Pub. Date: Jun. 1, 2006

(54) Dispenser having piezoelectric elements and method of operation

(76) Inventors: Mick A. Trompen, Westfield, IN (US); Gregory A. Lyon, Indianapolis, IN (US); Jeffery A. Meyer, Greenfield, IN (US); Steven D. Lubetkin, Zionsville, IN (US); Joseph R. Winkle, Carmel, IN (US); Ronald W. Lyon, Noblesville, IN (US)

Correspondence Address: Intellectual Property Group;Bose McKinney & Evans LLP, 2700 First Indiana Plaza, 135 North Pennsylvania Street, Indianapolis, IN, 46204, US

(21) Appl. No.: 11/226,479

(22) Filed: Sep. 14, 2005

Related U.S. Application Data
(63) Continuation-in-part of application No. 10/404,274, filed on Apr. 1, 2003.

(51) Int. Cl. A61M 37/00

(52) U.S. Cl. 604/131

(57) ABSTRACT

A dispenser is provided. The dispenser has a reservoir for storing a liquid or solid material. The dispenser may have a piezoelectric valve or pump located in an opening of the reservoir for regulating dispensing of the material. Alternatively, the dispenser may have a piezoelectric inchworm that directly or indirectly propels the material against or out of the opening. The opening may deposit the material directly or indirectly on the surface of an animal, or under the animal's skin. The dispenser also has means for being attached to the surface of the animal. Alternatively, the dispenser may be placed inside the animal. In certain embodiments, the dispenser comprises a reservoir containing a material to be dispensed, an actuable dispensing element, and an identifying device for remotely communicating information about the dispenser. The dispenser may also include a positioning device adapted to transmit the position of the dispenser.

### RELATED APPLICATIONS

This application is a continuation-in-part of U.S. patent application Ser. No. 10/404,274, filed on Apr. 1, 2003, the disclosure of which is expressly incorporated herein by reference.

This application is also related to a commonly assigned application entitled “Dispenser,” filed on even date herewith, the disclosure of which is expressly incorporated herein by reference.

### FIELD OF THE INVENTION

The invention relates to a dispenser for small quantities of liquids or solids, in particular a dispenser having one or more piezoelectric elements regulating the rate of dispensation.

### BACKGROUND OF THE INVENTION

Currently there are various methods used in control of pests on large domestic animals such as livestock. Commonly employed methods of pest control include ear tags or tapes, sprays and dusts, and back rubbers and dust bags.

Ear tags or tapes are pesticide-impregnated materials which are attached to animals by piercing the ear with a sharp post and corresponding locking receptacle or with adhesive. The pesticide slowly leaches from the carrier material and is deposited to the animal. The animal will then spread the material by moving its head from side to side and by rubbing alongside other animals.

This method of pesticide application tends to have a diminishing effect through its life cycle as the pesticide level eventually runs low enough that sub-lethal amounts of pesticide are deposited on the animal. This poses a serious problem, as sub-lethal applications of pesticide will allow pests to build a tolerance to the pesticide. Future generations of pests may subsequently become immune to what were once lethal exposures to the pesticide.

Sprays, dusts, and pour-on applications of pesticide involve the manual application of pesticide to the animal's back. Although this method can be effective, it requires additional herding and handling and may not be practical for large ranches or for free-range cattle.

Back rubbers and dust bags impregnated or filled with pesticide and suspended in a pasture in proximity to a salt lick, water supply or place where the animals are known to rest. The animals will make contact with the device in the normal course of their routine. In addition, the dust bag or back rubber may be located in a gateway which leads to a salt lick or water supply and which forces the animal to contact the device. Again, this method of insecticide application can be effective, but may not be practical for certain situations such as large ranches or free-range cattle.

It would be desirable to have an application method and dispenser that is capable of dosing a repeatable, prolonged, and lethal application of pesticide to an animal with no gradual decline, but rather, a sudden and complete cessation of exposure, and does not require further herding, handling or contact with the animal.

### BRIEF SUMMARY OF THE INVENTION

In one exemplary embodiment, the present invention provides a method of managing a group of animals. The method provides a plurality of dispensers, each dispenser containing a substance to be dispensed, a positioning device and an identification device. Each one of the identification devices is programmed with an identifier for a corresponding one of the animals, and each one of the dispensers is attached to the corresponding one of the animals and communicably linked to a monitoring station.

In another exemplary embodiment, a dispenser for dispensing a material is provided in accordance with the present invention. The dispenser comprises a reservoir containing a material to be dispensed, an actuable dispensing element located adjacent the reservoir or in the reservoir, an attachment member for attaching the dispenser to an animal, and an identifying device for remotely communicating information about the dispenser. The dispenser further comprises a positioning device adapted to transmit the position of the dispenser.

In yet another exemplary embodiment according to the present invention, a dispenser for dispensing a material comprises an attachment member for attaching the dispenser to an animal, a material to be dispensed contained within the container, and a remotely actuable dispensing element disposed about the dispenser.

### BRIEF DESCRIPTION OF THE DRAWINGS

The above-mentioned aspects of the present invention and the manner of obtaining them will become more apparent and the invention itself will be better understood by reference to the following description of the embodiments of the invention taken in conjunction with the accompanying drawings, wherein:

FIG. 1 is an exploded view of a first embodiment of the invention;

FIG. 2 is a side view of a second embodiment of the invention;

FIGS. 3-7 are cross sectional views of a third embodiment of the invention;

FIGS. 8-10 are cross sectional views of a fourth embodiment of the invention;

FIGS. 11-13 are cross sectional views of a fifth embodiment of the invention;

FIG. 14 is a perspective view of the first embodiment;

FIG. 15 is an exploded view of the lower section of the first embodiment;

FIG. 16 is a perspective view of yet another embodiment of a dispenser in accordance with the present invention; and

FIG. 17 is a schematic/diagrammatic view illustrating a method of managing a group of animals in accordance with the present invention.

Corresponding reference characters indicate corresponding parts throughout the several views.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

The device has a reservoir containing a material to be dispensed. The device also has one or more piezoelectric elements that act as a pump or as a valve to deliver prescribed and discrete measures of liquid or solid material. The piezoelectric elements may work in concert with other materials such as metal, plastic, ceramics, composite materials, etc. to form the complete pump/valve assembly. In addition, the piezoelectric element is coupled with an electronics array to control dose timing and actuation frequency. The electronics array will control dose timing and actuation frequency by controlling the supply of electrical energy to the piezoelectric element. There are several known methods for applying piezoelectric elements to fluid/liquid transfer, such as those used in some types of inkjet printers.

The dispenser may be designed to be attached to the surface of an animal, typically a domestic animal, in which case it will include an attachment member for maintaining the dispenser in position. Attachment members such as tissue piercing posts and grommets, tapes, adhesives, collars, harnesses, clamps, and staples or other such attachment members may be used.

The reservoir may be designed as a permanent component of the overall assembly or it may be removable and replaceable. In either case, the piezoelectric pump/valve will be coupled to the reservoir and the pesticide or other material will be transferred from the reservoir to the exterior of the device and eventually be deposited on the animal. The piezoelectric element may be adjacent to the reservoir or in the reservoir including in an opening of the reservoir.

The reservoir may include a one-way valve to allow air to enter the system as the pesticide or other material is transferred from the reservoir. In another embodiment, the reservoir may include an open cell foam sponge which will serve to keep the system primed by means of capillary action. The sponge will work in concert with a vent which is open to atmosphere. This technology is commonly applied to inkjet printing cartridges. In another embodiment, the reservoir may include a collapsible, flexible bladder to avoid drawing a vacuum as the pesticide or other material is transferred from the reservoir. The bladder design will also eliminate the entry of air to the liquid, which may disrupt the transfer of the pesticide or other material from the reservoir. Similarly, the reservoir may be designed along the lines of a syringe to accomplish the same task. As the pesticide or other material is transferred from the reservoir, the syringe piston will move to maintain a constant and predictable system pressure without the introduction of air to the liquid. In another embodiment, the device may dispense under vacuum. In this embodiment the piezoelectric pump or valve may be capable of dispensing the liquid as the system pulls a vacuum condition, eliminating the need for venting the reservoir.

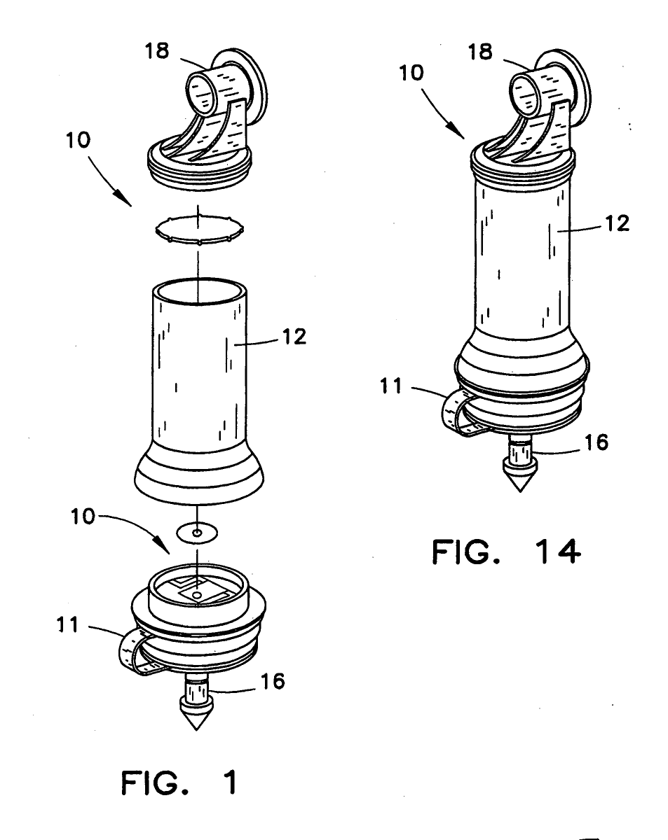

In one embodiment the piezoelectric element is a valve or pump located in an opening of the reservoir. FIG. 1 shows an example of this embodiment. The dispenser 10 has a reservoir 12 containing the material to be dispensed and a piezoelectric pump or valve 14 located inside to regulate the flow of the material being dispensed. The embodiment illustrated in FIG. 1 has a member for attaching the dispenser to the surface of an animal. In this case, the member comprises a post 16 which is meant to be removed from the device and placed through a piercing in the animal, for example in the animal's ear. Tape 11 in this embodiment is a nonconductive tape that covers one of the terminals of an internal battery. The tape is attached to post 16 so that when the post is removed from the device, the tape is also removed, allowing the internal battery to supply power to the piezoelectric element. After the post is passed through the piercing, locking ring 18 receives the end of the post to secure the dispenser in place.

FIG. 15 shows the lower section of the device 10 in an exploded view. Tape 11 is shown separated from post 16. The post is fit to battery cover 23 when the device is assembled. Contacts 19 connect battery 13 to electronics 15. As mentioned above, when post 16 is removed, tape 11 is also removed, allowing a circuit between battery 13 and electronics 15 to be completed. The electronics control piezoelectric element 14, which has piezoelectric contacts 25 and piezoelectric stack 27. The electronics are contained in and the piezoelectric element is mounted on electronics housing 27. Absorbent material 21 is also mounted on the electronics housing in the completed device. O-ring 17 allows a tight seal between the battery cover and the electronics housing.

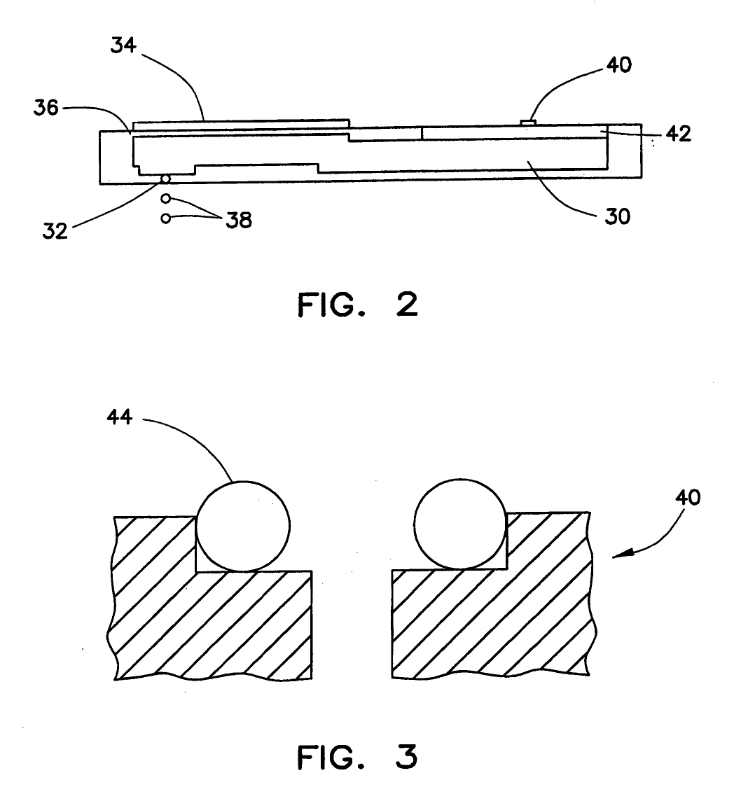

In one embodiment the piezoelectric element is located in or adjacent to either the reservoir or a chamber in fluid communication with the reservoir. By pulsing current through the piezoelectric element it is possible to expand the element, thereby reducing the space in the reservoir or chamber and forcing a small amount of liquid out of the reservoir or chamber. By pulsing current at a high frequency it is possible to expel a large number of droplets of fluid from the opening. FIG. 2 shows an example of this embodiment. The dispenser has a reservoir 30 having an opening 32. Piezoelectric element 34 expands and contracts as voltage is pulsed through it, causing it to press and release against flexible wall 36. This causes droplets or particles of material 38 to pass through opening 32. The illustrated embodiment also shows optional inlet 40 and optional filter 42. An additional container holding material to be dispensed may be connected to reservoir 30 via inlet 40. In such cases it may be desirable to interpose filter 42 between the inlet 40 and the opening 32 to prevent opening 32 from being clogged by environmental contamination.

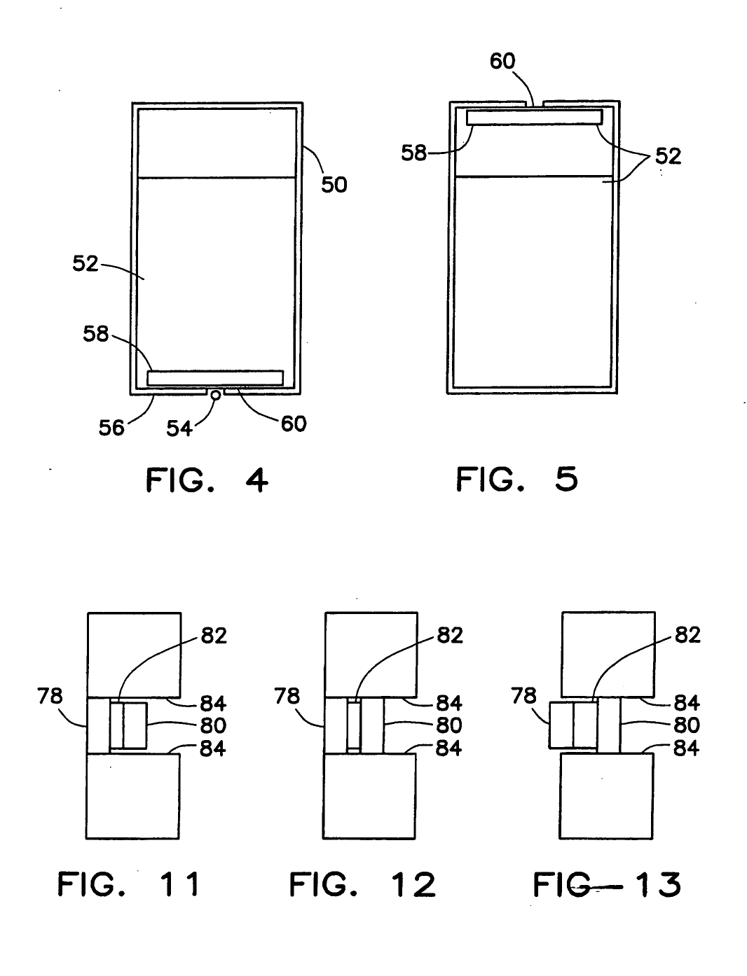

FIG. 3 shows a close-up detail of inlet 40. The inlet would typically contain an o-ring 44 to firmly seat an additional container. FIGS. 4 and 5 are schematic diagrams of a an additional container suitable for use in the present invention. The container 50 contains material 52 to be passed into the reservoir. The container has an opening 54 in wall 56 with means to couple to inlet 40 of dispenser 10. The illustrated container also contains an internal capillary action means consisting of an optional plate or filter 58 disposed adjacent to wall 56, defining a capillary channel 60 between the plate or filter 58 and the wall 56. The optional plate or filter or disc 58 may be used when the material 52 is a liquid material. This capillary channel ensures that if the container is inverted so that the material falls away from opening 54, a small amount of the liquid material will remain in the capillary channel 60. This is useful to reduce the likelihood of reservoir 12 emptying of material and becoming filled with air, as such an event could disrupt the proper pumping function of the piezoelectric element. In another embodiment, a different internal capillary action means could be used, such as a series of grooves or small passages leading directly to the inlet orifice of the piezoelectric element.

The device may also incorporate piezoelectric inchworm technology to eject a material from an element such as a syringe. In addition, the inchworm device could be used to advance a solid material through the end of a tube or similar device. Piezoelectric inchworm devices are comprised of several piezoelectric elements, which work together to produce a mechanical movement of the piezoelectric elements in a tube or along a rod or similar component. Alternatively, the piezoelectric elements may remain stationary while causing movement of another element such as a rod.

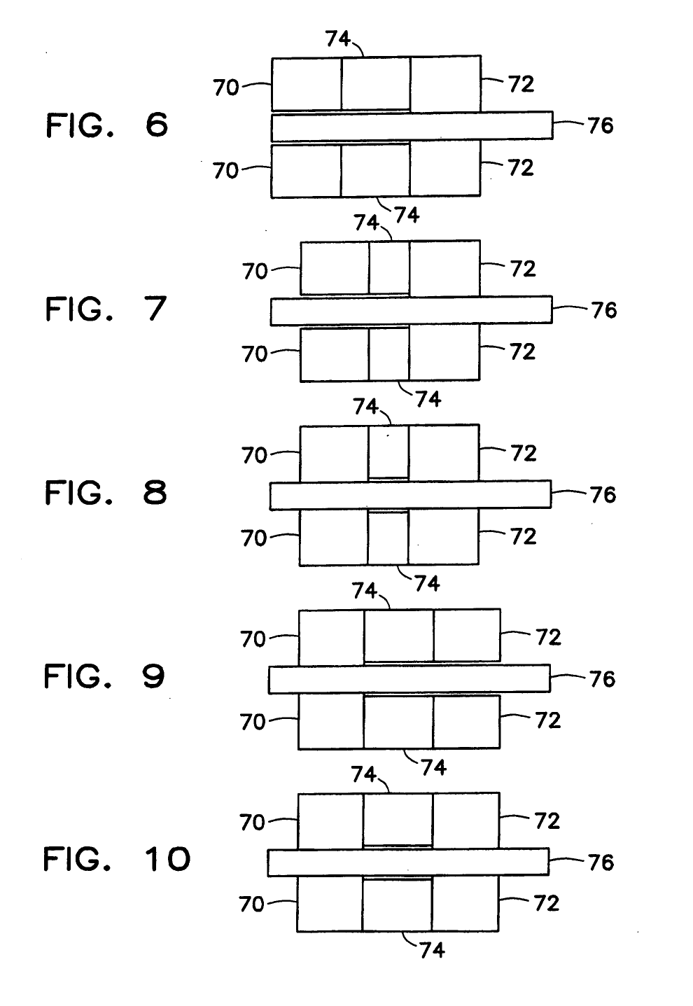

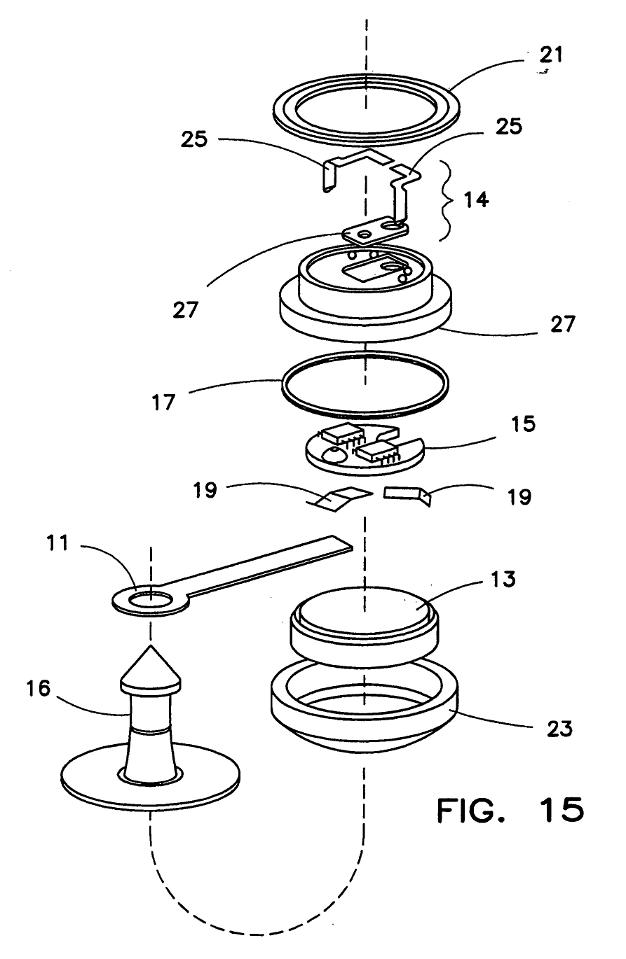

FIGS. 6 through 10 illustrate the operation of an inchworm device having ring-shaped piezoelectric elements 70, 72, 74 shown in cross-section operating to move the inchworm along a rod 76. Elements numbered 70 and 72 expand and contract radially with respect to the rod, while elements 74 expand and contract axially with respect to the rod. In FIG. 6 elements 70 are contracted, elements 74 are expanded, and elements 72 are expanded to clamp against rod 76. FIG. 7 demonstrates the next step, in which elements 74 contract, pulling elements 70 toward elements 72. FIG. 8 shows the succeeding step, in which elements 70 expand to clamp against rod 76. FIG. 9 shows the assembly after elements 72 contract, releasing their grip on rod 76, and elements 74 expand, moving elements 72 away from elements 70. In FIG. 10 elements 72 once again expand to clamp on rod 76. The cycle repeats as elements 70 contract to release their grip on the rod, resulting in the situation as shown in FIG. 6. Of course, if either elements 70 or 72 were fixed in place against a stationary surface, then the same process could be used to impart axial movement to a movable rod rather than moving the inchworm along a fixed rod. Furthermore, it would be possible to operate the inchworm using a set of piezoelectric elements adjacent to a rod, rather than ring-shaped elements as shown in FIGS. 6 through 10.

FIGS. 11 through 13 show another embodiment of the inchworm, in which the inchworm has piezoelectric elements 78, 80, and 82 which move through a tubular space defined by walls 84. Elements 78 and 80 expand and contract radially to fit against the walls 84 of the space, while element 82 expands axially along the direction of movement. FIG. 11 shows element 78 expanded against walls 84, element 80 contracted to move freely, and element 82 contracted. FIG. 12 shows the situation after element 80 expands to fit against walls 84, and FIG. 13 shows the situation after element 78 contracts to release its grip on walls 84 and element 82 expands to move element 78 away from element 80. Following these steps, element 78 would expand again, element 80 would contract to release its grip on walls 84, and element 82 would contract again, at which point the assembly is returned to the configuration shown in FIG. 11, but displaced in the direction of travel.

In another embodiment, the device may be entirely or partially implanted in an animal in a known manner in order to dose pesticides, therapeutic agents, growth hormones, medicines, drugs, etc. If the animal is a ruminant, the entire device may also be deposited to the animal's rumen with a balling gun. Intraruminal devices for dispensing drugs, medicine, hormones, etc. are disclosed in co-pending application Ser. No. 10/141,300, the contents of which are incorporated herein in their entirety. Such devices generally contain a housing with externally mounted retention device such as a pair of plastic “wings”, or else are weighted to prevent their being passed out of the rumen. The device may also be attached to the animal with only a small portion of the device implanted under the skin of the animal to act as a passage for the dosing of a desired material.

The device can dose a discrete amount of material on demand, on a prescribed timed interval or it may dose continuously and indefinitely over an extended time period until the insecticide or other material is exhausted.

In addition, since the primary delivery is not through diffusion and is not dependent on high solubility of a parasiticide in a polymer matrix, the fluid utilized in the present invention can be expanded to include previously unusable parasiticides and still include compounds previously dissolved in polymer matrices. These include, but are not limited to various avermectins, benzimidazoles, milbemycins, carbamates, organophosphates, phenylpyrazoles, amidines, insect growth regulators, juvenile hormones, nicotinoids, pyrroles and naturalytes (i.e., the spinosyn family). Representative compounds may include abamectin, doramectin, eprinomectin, selamectin, alphamethrin, amitraz, coumaphos, ivermectin, deltamethrin, cyhalothrin, diazinon, cyromazine, cypermethrin, milbemycin, cyfluthrin, cyloprothrin, famphur, fenthion, fenvalerate, flucythrinate, flumethrin, fipronil, hexaflumaron, imidacloprid, lindane, lufenuron, malathion, methoprene, metriphonate, moxidectin, pernethrin, pyrethrin, pyrethrum, phomet, pirimiphos, chlorvinphos, rotenone, propetamphos, tetrachlorvinphos, zetacypermethrin, chlorpyrifos and spinosad, among others.

In another embodiment, the actuation of the device may be controlled by a centrally located RF (radio frequency) transponder. Each device in this embodiment will incorporate a RF receiver which will allow an operator to transmit instructions to the electronics array. This RF technology could be used to deliver insecticide or other materials on demand or it could be used to change the program, dose frequency, dose amount, rate of delivery, etc. It would also be possible to use the RF signal as the power source for the device, thus greatly reducing the weight by avoiding the use of batteries. In further embodiments, the receiver may also transmit a signal to a remotely actuable dispensing element about the dispenser. In addition to radio frequency signal transmissions, the signal may also be delivered to the device by way of cellular communications, satellite signals, infrared radiation or the like.

The device may be activated initially by the user in a number of ways. In one embodiment, the user may remove an insulating strip located between an electrical contact and battery or between two electrical contacts. The strip may be removed by attaching it to an integral component of the device, such as a piercing post, such that any attempt to use the post to mount the device would necessarily force the user to remove the strip. The strip may also be attached to the packaging of the device. As the device is removed from the package, the strip is pulled out and the device is activated. In another embodiment, the device may be activated with a push button or a similar device. In another embodiment, the device may be delivered to the user with an integral part, such as the piercing post, inserted into the device, such that the integral part holds a circuit in the open position. When the user removes the integral part prior to making use of the device, the circuit would close. In the alternative, the part may make momentary contact with a circuit to initiate a pre-programmed response of activating the device.

In another embodiment, a photovoltaic cell or similar device may be used to activate the device. The device may be activated as it is removed from its packaging and exposed to light. The device may be activated as a strip covering the photovoltaic cell or similar device is removed and exposed to light. The device may be activated as an integral component such as the male piercing post is removed from a section of the device to attach the device to an animal. The piercing post could be situated such that it covers the photovoltaic cell until it is removed for use.

The device may be activated as the piercing post is seated to the retention grommet. This action may complete a circuit or break a circuit or make momentary contact to initiate activation. The device may also be activated with a magnetic reed switch and a magnetic application tool.

The device may be powered with a battery or photovoltaic device and a voltage converter. In another embodiment, the device could be powered with another piezoelectric element such as a bender. The bender could be actuated through movement from the animal or from the animal's pulse and the produced voltage could be stored. The stored energy could then be used to power the piezoelectric element and associated electronics that control the pump/valve assembly. In another embodiment, the device could be powered by the animal's body temperature and a thermoelectric device or thermoelectric pile to generate a current. The produced current could be stored. The stored energy could then be used to power the piezoelectric element and associated electronics that control the pump/valve assembly.

In another embodiment, the device could be powered with a device similar to a self-winding watch mechanism where the mechanical energy derived from the concentric motion of the device is converted to electrical energy and stored. The stored energy could then be used to power the piezoelectric element and associated electronics that control the pump/valve assembly.

The pesticide or other material may be transferred directly to the surface of the animal, to its skin or fur. Another embodiment would direct the pesticide or other material to an absorbent material, sponge, felt, cloth or other absorbent or porous material in proximity to the animal's skin or fur. The material will be such that it can retain the liquid pesticide or other material without a measurable or significant portion being released in droplet form and falling to the ground. Through the animal's normal movements, the liquid would be rubbed off to the animal's skin or fur. In another embodiment, the external surface of the device would have an external capillary action means such as a series of grooves molded adjacent the outlet of the piezoelectric pump or valve. These grooves would capture and retain the expelled liquid on the exterior surface of the device, allowing it to eventually contact the animal. The capillary action means may be used in conjunction with the absorbent material, or by itself. Another embodiment would direct the pesticide or other material to a secondary and exterior reservoir or surface of the device, which would allow the insecticide to collect or pool and eventually roll onto the animal. The secondary reservoir or surface will be located such that there is a high probability that the collected pool of pesticide or other material will make contact with the animal and not be lost by dropping to the ground.

Once the pesticide or other material has reached the animal, it is spread further through the animal's normal movement and interaction with other animals. As the animal moves it head from side to side and rubs along other animals, the pesticide or other material is spread along the entire length of the animal.

While the device has been discussed in the context of large domestic animals such as ruminants, it would be equally suitable for use with smaller domestic animals, such as companion animals.

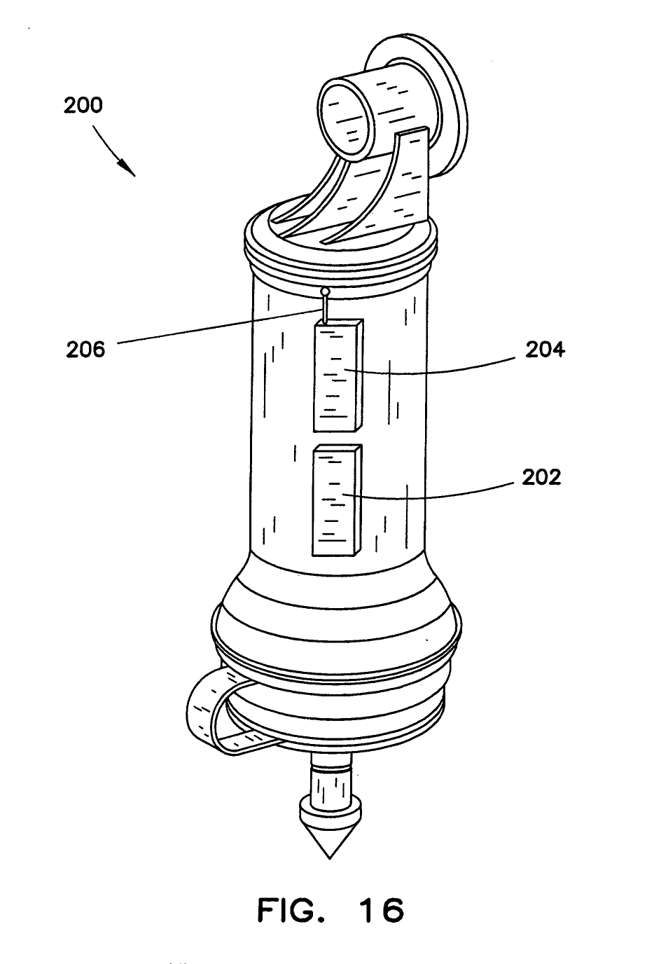

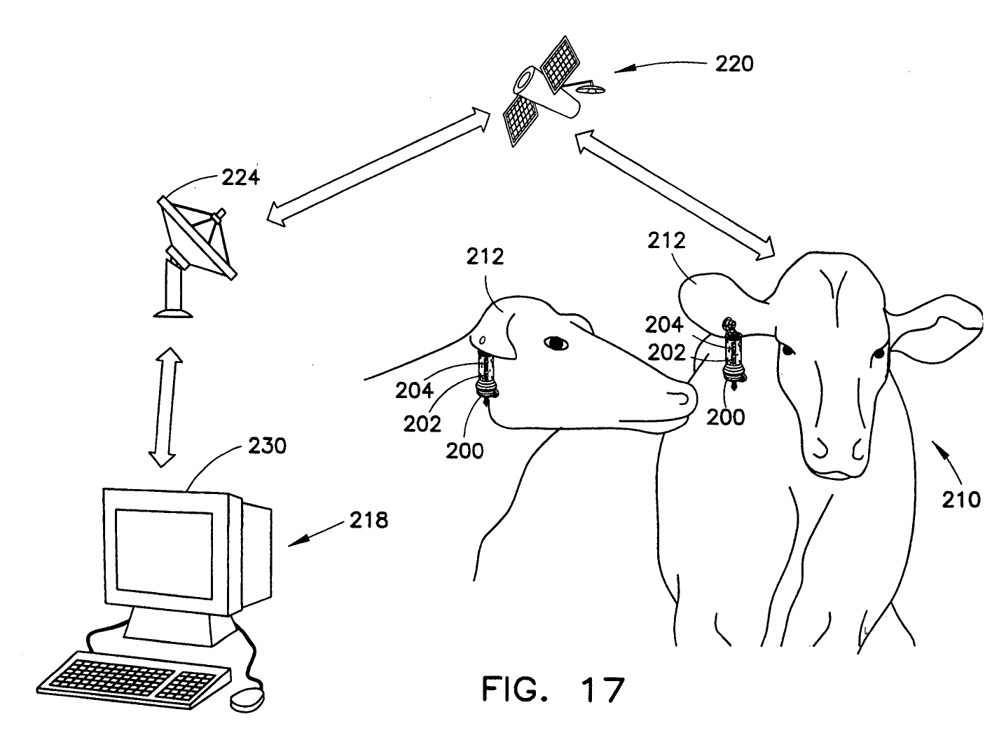

Dispensers embodied by the present invention may also be equipped with identification and tracking or positioning devices. FIG. 16 schematically shows a dispenser 200 that includes identification device 202, which is provided as a radio frequency identification (“RFID”) tag. RFID tags are known in the art and their general operation need not be described herein. The RFID tag can be programmed with various identifying information about the particular animal wearing dispenser 200, such information including the name or identifying number of the animal, its birth date, weight, sex, lineage and other identifying characteristics. Of course, FIG. 16 merely schematically represents an RFID. In fact, the RFID may be implanted in the animal rather than contained on or in dispenser 200. In any event, the RFID 202 would be communicably linked to other electronics in the dispenser, as described in more detail below.

Dispenser 200 also includes a tracking device 204 that preferably includes a global positioning system (“GPS”) device and a communications device having antenna 206 that allows the communications device to transmit a signal to another device. For example, it is known in the art to combine cellular technology with GPS devices such that signals can be transmitted from the GPS device through the nearest cellular tower, from where the signal can be conventionally transmitted to a center for processing. In this manner, the location and other data contained in device 204 of a particular animal wearing dispenser 200 can be transmitted to a central location and monitored. Further, tracking device 204 may also include a receiver, such as the RF receiver described above, making device 204 a two-way communications device. An actuation signal can be sent to the receiver to dispense on demand, such that the dispenser can be remotely controlled.

In general, then, one of skill in the art would appreciate from these teachings that dispenser 200 can be equipped with a two-way communications device(s) as well as a GPS device or other tracking/positioning device that determines the location of dispenser 200. The dispenser may also be equipped with temperature, humidity, and other detection devices that can be transmitted to a monitoring station. These features when coupled with the inventive dispenser allow for a wide range of systems to manage a group of animals.

For example, FIG. 17 illustrates a system for managing a group of animals. Cows 210 have dispensers 200 attached to their ears 212 via an attachment member. Each dispenser contains a tracking device 204 and an identification device 202. The tracking device also includes a communications device, such as the RF receiver described above, so that information can be transmitted between the dispenser and base station 218. As described above, each identification device is programmed with identifiers for the particular animal to which device 200 is attached. Through satellite 220, the position of each dispenser is known. Signals from satellite 220 can be received by a satellite dish 224 and in turn conveyed to a computer network and computer station 230. Alternately, the position information received by tracking device 204 can be transmitted by conventional means, such as a cellular communications network, and then relayed to a computer network and monitored at computer station 230.

Still referring to FIG. 17, as a specific example, the dispenser 200 may be provided with a substance that is desirable to dispense at only appropriate times, such as when the outside temperature reaches a certain level. In this event, a person operating computer station 230 may remotely send a signal to one of more of the dispensers 200 to actuate the dispensers to dispense at least a portion of the substance when the temperature reaches the predetermined level. Dispenser 200 may be equipped with a temperature sensor that transmits a temperature signal to computer station 230.

In another specific example, it is desirable to mate two particular cows. Monitoring station 230 can be employed for this task, tracking the particular two cows as described above. Once the monitoring station (or the person observing the cows' activity at the monitoring station) determines that the animals are sufficiently close, a signal can be sent to dispense a pheromone from one of the dispensers 200, thereby inducing the two animals to mate. One of ordinary skill in the art would readily recognize many other selection criteria for remotely activating dispenser 200. The dispenser can be configured with a simple binary switch coupled to the dispensing element and which can be remotely activated. Similarly, the dispenser can be equipped with more sophisticated electronics that would allow remotely controlling, e.g., dose size, dose frequency, etc., as described above. The remotely actuable dispensing element is disposed about the dispenser and may comprise a piezoelectric element like that described in detail above. Alternatively, one of skill in the art may adapt the novel methods just described with dispensers having other remotely actuable dispensing elements.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method of managing a group of animals, comprising:
(a) providing a plurality of dispensers, each dispenser containing a substance to be dispensed, a positioning device and an identification device;
(b) programming each one of the identification devices with an identifier for a corresponding one of the animals;
(c) attaching each one of the dispensers to the corresponding one of the animals; and
(d) communicably linking each one of the dispensers to a monitoring station.

2. The method of claim 1, further comprising sending a signal from the monitoring station to at least one of the plurality of dispensers, the signal causing the one dispenser to dispense at least a portion of the substance.

3. The method of claim 2, further comprising sending the signal to more than one of the plurality of dispensers.

4. The method of claim 1, wherein the substance comprises a pesticide.

5. The method of claim 1, wherein the group of animals is comprised of cows.

6. The method of claim 1, further comprising communicating the position and the identity of one of the animals to the monitoring station.

7. The method of claim 6, further comprising communicating a signal from the monitoring station to the dispenser of the one animal based upon the animal's monitored position.

8. The method of claim 1, further comprising evaluating at the monitoring station position and identifier data of the animals and sending a signal to one of the dispensers based upon the evaluating.

9. The method of claim 8, wherein the signal is a dispense signal.

10. The method of claim 8, wherein the signal causes the one dispenser to change dose frequency, dose amount or rate of delivery of the substance to be dispensed.

11. The method of claim 1, wherein the identification device comprises an RFID.

12. A dispenser for dispensing a material, comprising:
a reservoir containing a material to be dispensed;
an actuable dispensing element located adjacent the reservoir or in the reservoir;
an attachment member for attaching the dispenser to an animal; and
an identification device adapted to remotely communicate information about the dispenser.

13. The dispenser of claim 10, further comprising a positioning device adapted to transmit the position of the dispenser.

14. The dispenser of claim 13, wherein the positioning device comprises a GPS device.

15. The dispenser of claim 12, wherein the dispensing element comprises a piezoelectric element.

16. The dispenser of claim 12, wherein the material comprises a pesticide.

17. The dispenser of claim 12, wherein the dispenser comprises a remotely accessible switch adapted to actuate the dispensing element.

18. The method of claim 12, wherein the identification device comprises an RFID.

19. A dispenser for dispensing a material, comprising:
a reservoir containing a material to be dispensed;
an actuable dispensing element located adjacent the reservoir or in the reservoir;
an attachment member for attaching the dispenser to an animal; and
a positioning device adapted to transmit the position of the dispenser.

20. The dispenser of claim 19, further comprising an identification device for remotely communicating information about the dispenser.

21. The method of claim 20, wherein the identification device comprises an RFID.

22. The dispenser of claim 19, wherein the positioning device comprises a GPS device.

23. The dispenser of claim 19, wherein the dispensing element comprises a piezoelectric element.

24. The dispenser of claim 19, wherein the material comprises a pesticide.

25. The dispenser of claim 19, wherein the dispenser comprises a remotely accessible switch adapted to actuate the dispensing element.

26. A dispenser for dispensing a material, comprising:
an attachment member for attaching the dispenser to an animal;
a material to be dispensed contained within the dispenser;
a remotely actuable dispensing element disposed about the dispenser; and
a receiver adapted to receive and transmit a signal to the remotely actuable dispensing element.

27. The dispenser of claim 26, further comprising a positioning device adapted to transmit the position of the dispenser.

28. The dispenser of claim 27, wherein the positioning device comprises a GPS device.

29. The dispenser of claim 26, further comprising an identification device for remotely communicating information about the dispenser.

30. The dispenser of claim 29, wherein the identification device comprises an RFID.

31. The dispenser of claim 26, wherein the dispensing element comprises a piezoelectric element.

32. The dispenser of claim 26, wherein the material comprises a pesticide.

33. The dispenser of claim 26, wherein the signal is adapted to cause the dispenser to change dose frequency, dose amount or rate of delivery of the material to be dispensed.
